(19) United States
(12) Patent Application Publication
(10) Pub. No.: US 2024/0351038 A1
(43) Pub. Date: Oct. 24, 2024

(54) DEVICE AND METHOD FOR LABELING SAMPLES BY THE SPLIT AND POOL APPROACH

(71) Applicant: Scale Biosciences Inc., San Diego, CA (US)

(72) Inventors: Frank Steemers, Encinitas, CA (US); Fan Zhang, San Diego, CA (US); Kim-Xuan Nguyen, Fremont, CA (US); John Haliburton, Redwood City, CA (US); Garry P. Nolan, Redwood City, CA (US); Baxter Eldridge, Piedmont, CA (US)

(21) Appl. No.: 18/569,198

(22) PCT Filed: Jun. 23, 2022
(86) PCT No.: PCT/US2022/034798

§ 371 Date: Dec. 11, 2023

Related U.S. Application Data
(60) Provisional application No. 63/214,731, filed on Jun. 24, 2021.

(51) Int. Cl. B01L 3/00

(52) U.S. Cl. CPC B01L 3/5085; B01L 2200/025; B01L 2300/0829; B01L 2300/0864; B01L 2300/123; B01L 2400/0409; B01L 2400/0487

(57) ABSTRACT

This disclosure provides, among other things, a device for splitting and pooling a biological sample. In some embodiments, the device is capable of being switched back and forth between: (i) a pooling state in which some of the sample is pooled in one or more compartments; and (ii) a split state in which each pooled sample of (i) is split into multiple compartments. Various implementations of the device as well as a method of use are also provided. A device for collecting a sample from multi-well plate is also provided.

### CROSS-REFERENCING

This application claims the benefit of U.S. provisional application Ser. No. 63/214,731, filed on Jun. 24, 2021, which application is incorporated by reference herein for all purposes.

### BACKGROUND

Index sequences (also known as “barcode” sequences) can be added to targets (e.g., cells and cellular components, including DNA, RNA and protein, as well as beads, etc.) using a “split and pool” approach. Such methods may involve partitioning the targets into compartments (such that each compartment receives multiple targets), adding a subunit for an index to the targets while they are compartmentalized (where each compartment may receive a different subunit), pooling the targets, and then repeating the process as many times as desired. If the partitioning is random, then each target should have received a different set of subunits, and therefore a different index, by the end of the process. In some cases, the targets are barcoded such that the pool of targets can be statistically subsampled with unique, different indices. Because the total number of indexing combinations grows exponentially with the number of the split and pool cycles, the split and pool approach can be used to index an almost unlimited number of targets. Examples of split and pool methods for labeling proteins and RNAs on a cell-by-cell basis are described in O'Huallachain et al (Commun. Biol. 2020 3: 279) and Cao et al (Science. 2017 357: 661-667), among many others.

One problem with the way in which split and pool methods are typically performed is that the pooling and partitioning steps require a significant number of liquid transfer steps. For example, pooling and re-partitioning a 96-well plate to a single tube and back again requires 96 individual transfers into the tube, then another 96 transfers back. In theory, a conventional liquid handling robot could be used for each of the steps. However, the non-parallel nature of split and pool approach makes such conventional robots less than ideal. Additionally, liquid transfer steps can lead to loss of sample, which can cause problems in some cases.

This disclosure provides a solution to this problem.

### SUMMARY

This disclosure provides, among other things, a device for splitting and pooling one or more biological samples. In some embodiments, the device is capable of being switched back and forth between: (i) a pooling state in which a sample split between multiple compartments becomes pooled; and (ii) a splitting state in the which the sample pooled in (i) becomes split into multiple compartments. This device is pipette-free and can be switched between the pooling state and the splitting state without a liquid handling workstation. A device for collecting a sample from multi-well plate is also provided.

Various implementations of the device as well as methods of use are described below.

### BRIEF DESCRIPTION OF THE FIGURES

Some aspects of the technology described herein may be best understood from the following detailed description when read in conjunction with the accompanying drawings. It is emphasized that, according to common practice, the various features of the drawings are not to scale. Indeed, the dimensions of the various features are arbitrarily expanded or reduced for clarity. Included in the drawings are the following figures.

FIGS. 1A-1J illustrate an example of a “dam” plate. FIG. 1A shows a multi-compartment plate (left) in which the compartments are connected by channels, a close-up of four wells showing the channels (middle) and a close-up of a dam element that engages with the multi-compartment plate and blocks at least some of the channels (right). In this example, the dam element sits over the multi-compartment plate and contains openings that allow reagents and barcodes to be added to or removed from the multi-compartment plate, after the plate and dam element are engaged. FIG. 1B shows an example in which a dam element is inserted into a channel between two wells, thereby closing the channel. The channel can be opened by removing the dam element. FIG. 1C illustrates that, in a multi-compartment plate, each compartment does not need to be connected to all of the compartments next to it. FIG. 1D-1H illustrates further examples of a dam plate. Results obtained from use of one implementation of a plate that can be switched from splitting to pooling (or visa versa) by applying a force from the bottom of the well are shown in FIG. 1I and FIG. 1J.

FIGS. 2A-2D illustrate an example of a “flexible” plate. FIG. 2A illustrates how a stretchable membrane can be stretched from its original state (left) to a state that has compartments (right). FIG. 2B illustrates how compartments can be produced by pushing projections into a flexible membrane that is on top of a template that contains openings. The projections can be pins, as shown, but other shapes could also be used. FIG. 2C illustrates a flexible membrane that is on top of a template that contains openings. Depending on how the device is configured, the membrane could be moved by pushing or pulling, and the membrane can be moved through the openings or away from the openings. FIG. 2D shows that compartments can be made by pulling the membrane, as opposed to pushing the membrane, using a plate that has projections. In any embodiment, the membrane can be affixed, e.g., bonded, to the template.

FIG. 3 illustrates an arrangement of linear connected compartments. In the embodiment shown in FIG. 3, the compartments that are fluidically connected in series. In this example, there are two pooling reservoirs (syringes) that are connected to the compartments at the ends. As indicated below, a pressure driven pump could also be used.

FIG. 4A-E illustrate examples of a collection plate. FIG. 4A shows a side view of an example of the collection plate. FIG. 4B shows an oblique view of an example of the collection plate. FIG. 4C shows an example of a collection plate with sloped walls and frame for standing.

FIG. 4D shows the top and bottom view of the collection plate, including interior and exterior areas. FIG. 4E illustrates a collection plate with a foldable skirt.

FIG. 5 shows an example workflow on how the plate can be used for pooling and collection.

FIG. 6 shows an example on how the plate can be used for collecting and washing of cells or nuclei.

FIG. 7. shows the pictures of an actual manufactured device used in some examples.

### DETAILED DESCRIPTION

Before embodiments of the present invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a nucleic acid” includes a plurality of such nucleic acids and reference to “the compound” includes reference to one or more compounds and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

All patents and publications, including all sequences disclosed within such patents and publications, referred to herein are expressly incorporated by reference.

The headings provided herein are not limitations of the various aspects or embodiments of the invention. Accordingly, the terms defined immediately below are more fully defined by reference to the specification as a whole.

As noted above, a device for repeatedly splitting and pooling one or more biological samples is provided. The device is capable of being readily switched back and forth between a splitting state in the which a pooled sample becomes split into multiple compartments and a pooling state in which a sample that is split between multiple compartments becomes pooled. In the splitting state, the previously pooled sample is split into at least 2, least 4, at least 8, at least 16, at least 48, or at least 96 compartments. In the pooling state, the previously split sample is pooled into a smaller number of compartments. For example, in the pooling state, a sample that is split into n compartments (wherein n=at least 2, least 4, at least 8, at least 16, at least 48, or at least 96, etc.) may be pooled into a smaller number of compartments (e.g., n/2, n/4 or n/8 compartments). In some cases, in the splitting state, the previously pooled sample may be split into at least 8 compartments, whereas in the pooling state the sample (which was previously split between least 8 compartments) may be pooled into a single compartment. The device can be readily switched between the two states, thereby allowing a sample to be split and pooled as many times as needed, without the need for a pipette or a liquid handling workstation. In any embodiment, the compartments may have a volume in the range of 5 ul to 50 ml, 50 mL to 150 mL, 150 mL to 500 mL e.g., 10 ul to 500 ul, or 20 ul to 200 ul. Individual compartment volumes can range from 5 ul to 5 mL.

The device may be used in a variety of split and pool synthesis methods, including split and pool chemical synthesis methods that are done on beads or in solution (see, e.g., Halpin et al PLoS Biology 2004 2: 1015-1021 and Halpin, PLoS Biology 2004 2: 1022-1030).

In particular embodiments, the device may be employed to index analytes in one or more samples using a split and pool approach, the general goal of which is to add a unique index to analytes (e.g., proteins, RNA, DNA, cDNA etc.) that come from the same source (e.g., the same cell or nucleus), so that they can be distinguished from one another. For example, if the sample contains cells, then the index added to the analytes in a particular cell will be different to the index added to the analytes from other cells. In these embodiments, the term “index” is used to refer to a molecule that has a complexity that is sufficient to distinguish between the analytes of the different entities (cells or nuclei) and the term “subunit” is intended to refer to the building blocks for the index (e.g., nucleotides, oligonucleotides, chemical fluorophores, mass tags, etc.). In some embodiments, a subunit may be a unique molecular index or UMI, which may contain a random sequence of nucleotides (of a length of, e.g., 4-30 nucleotides), In any embodiment, an index can contain a unique combination of subunits. In the following description, when a subunit is “added” to a sample, the subunit is added to analytes (e.g., protein, RNA, DNA or cDNA, etc.) that are in the sample.

In some embodiments, the method may comprise (a) adding subunits of an index to a split sample that is present in the device and (b) switching the device to the pooling state, thereby pooling the split sample of step (b). In these embodiments, the split sample may be made by loading one more samples onto the device when the device is in the splitting state. For example, if there are 24 different samples, then the method may comprise adding the different samples to different compartments while the device is in the splitting state. In other embodiments, the split sample may be made by switching the device that contains a pooled sample from a pooling state to a splitting state, thereby splitting a pooled sample into multiple compartments.

In either embodiment, the method may comprise (c) allowing the pooled sample produced in (c) to mix (e.g., by rocking, shaking, or any other means), to allow the analytes (which were previously partitioned) to become mixed, and (d) switching the device to a splitting state, thereby splitting the pooled sample into multiple compartments. In these embodiments, the method may comprises repeating the subunit addition and pooling steps at least once (e.g., at least twice, 5 times or at least 10 times), each repeat followed by steps (c) and (d), optionally except for the final repeat, to produce an indexed sample. The sample may then be collected.

In any embodiment, unique indices may be added to the device during the split-pool process. Alternatively unique indices may be pre-loaded in the device.

In very general terms, split-pool methods involve partitioning a sample into several compartments, adding a different subunit (or “building block”) for the index to each partition, pooling the sample, then repeating the partitioning, addition and pooling steps as needed until a sufficient number of subunits have been added and the targets in the sample are uniquely indexed.

In any embodiment, the one or more samples may be applied to the device while it is in the splitting state, the pooling state, or in another state (e.g., a “loading” state). For example, in some embodiments, multiple samples may be applied to the device when it is in the splitting state, which each compartment receives a different sample. In other embodiments a single sample may be applied to the device when it is in the splitting state, which each compartment receives a different aliquot of the same sample. Alternatively, the sample can be applied to the device in its pooling state or loading state.

In any embodiment, the method may involve adding different subunits to the sample while the sample is split (typically one subunit per compartment, where the different compartments receive different subunits). The split sample is then pooled by switching the device into its splitting state. After pooling, the pooled sample may be mixed so that the entities (e.g., cells, particles or nuclei, etc.) that were previously in different compartments become intermingled with one another. After mixing, the sample can be split and the process repeated as many times as needed.

In any embodiment, the sample may be split into multiple compartments. After splitting, different subunits are added to the sample while the sample is split (typically one subunit per compartment, where the different compartments receive different subunits). Next, the method involves switching the device to the pooling state, thereby pooling the compartmentalized sample. The compartmentalization, addition, and pooling steps can then be repeated, as necessary, until the sample is indexed. In these embodiments, the method may comprise repeating the compartmentalization and addition steps at least once, e.g., multiple times, each repeat followed by a pooling step, except for the final repeat when the pooling step is optional. After these steps have been repeated, the indexed sample may be collected. As would be apparent, suitable mixing, addition, and/or washing steps may be performed in between or during any of the steps of the method. The general principles of how indexing can be done using the split and pool approach are described in a variety of publications including Kuchina et al (Science 2021 371:eaba5257), O'Huallachain et al (Commun. Biol. 2020 3: 279), Cao et al (Science 2017 357: 661-667) and Rosenberg (Science 2018 360: 176-182), among many others. These publications are incorporated by reference for their descriptions of how split and pool methods can be implemented. In any of these examples, single cells are uniquely indexed enabling single cell analysis.

As noted above, in some embodiments, the sample may comprise cells, nuclei or particles. In these embodiments, the cells or nuclei may be fixed and/or permeabilized prior to starting the method. The sample may contain cells that are in solution, e.g., cultured cells that have been grown as a cell suspension. The sample may contain analytes, (DNA, RNA, and protein) that are uniquely indexed using the split-pool process. In other embodiments, disassociated cells (which cells may have been produced by disassociating cultured cells or cells that are in a solid tissue, e.g., a soft tissue such as liver or spleen, etc. using trypsin or the like) may be used. In particular embodiments, the sample may contain blood cells, e.g., whole blood or a sub-population of cells thereof. Sub-populations of cells in whole blood include platelets, red blood cells (erythrocytes), platelets and white blood cells (i.e., peripheral blood leukocytes, which are made up of neutrophils, lymphocytes, eosinophils, basophils, and monocytes).

Depending on the nature of the subunit being added in the addition step (i.e., whether the subunit is subunit is an oligonucleotide, a unique molecular index, a nucleotide, a fluorescent dye, a mass tag or another chemical moiety), the addition may be catalyzed by chemical or physical means, e.g., enzymatically, e.g., by ligation or polymerase extension, by nucleic acid hybridization, or by an addition reaction. If the sample contains cells or nuclei, then the addition may be biorthogonal addition reaction (e.g., using click chemistry, for example). The index may be added to cDNA, RNA, protein, genomic DNA, genomic DNA, primer extension products, or ligation products, or oligonucleotides, or any combination of RNA, cDNA, protein or DNA, oligonucleotide attached to an analyte (e.g., antibody oligo conjugate), as desired, thereby allowing data from those molecules to be assigned to a particular cell.

Various implementations of the device described below.

### Maze/Dams Plate

In some embodiments, the device may comprise: (a) a multi-compartment plate (i.e., that contains at least 2, least 4, at least 8, at least 16, at least 48, or at least 96 compartments) in which the compartments are fluidically connected by channels and (b) a dam element that is adapted to engage with the multi-compartment plate and block at least some of the channels. In these embodiments, the device can be switched back and forth between the splitting state and the pooling state by engaging and disengaging the dam element with/from the multi-compartment plate, i.e., by inserting the dam element into the multi-compartment plate to block at least some of the channels and removing the dam element from the multi-compartment plate remove the dams.

Switching the device from the pooling state to the splitting state or vice versa can be implemented from the top or bottom of the plate. For example, dams can be added and removed from the top of the plate by fitting the projections of a dam element into the channels from above. In other embodiments, at least part of the plate (e.g., the bottom of the plate) may be made of a flexible material that can be stretched up or down to create the dam. In these embodiments, the dam element may push (or pull) the flexible material to create the dam. In these embodiments, the bottom of the well may be flexible, enabling switching between a temporary well or a flat or semi-flat surface that connects with a neighboring well. In some embodiments, a flexible part of the plate can be squeezed thereby temporarily closing or blocking the flow between wells. In these embodiments, the dam element can be engaged with the multi-compartment plate and removed to split and pool the sample as many times as desired.

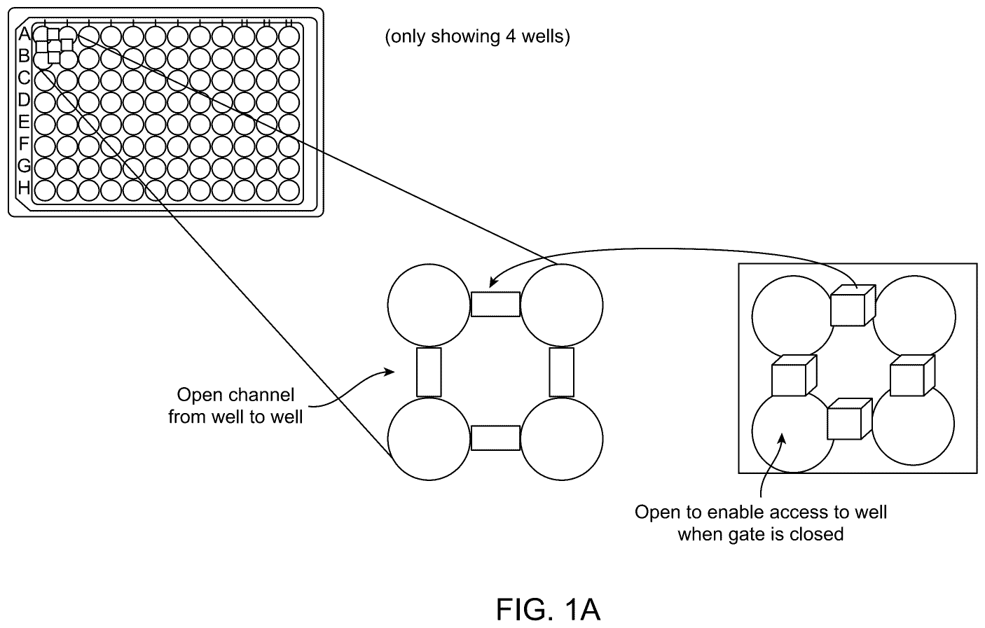

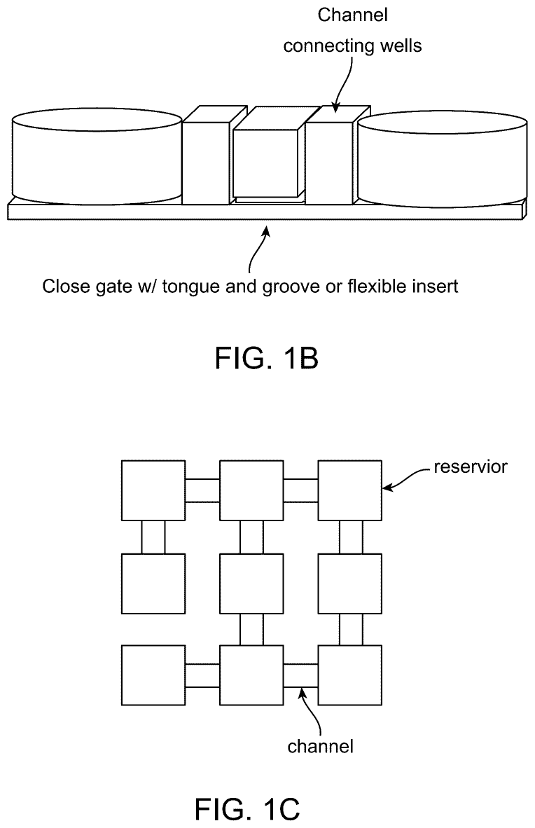

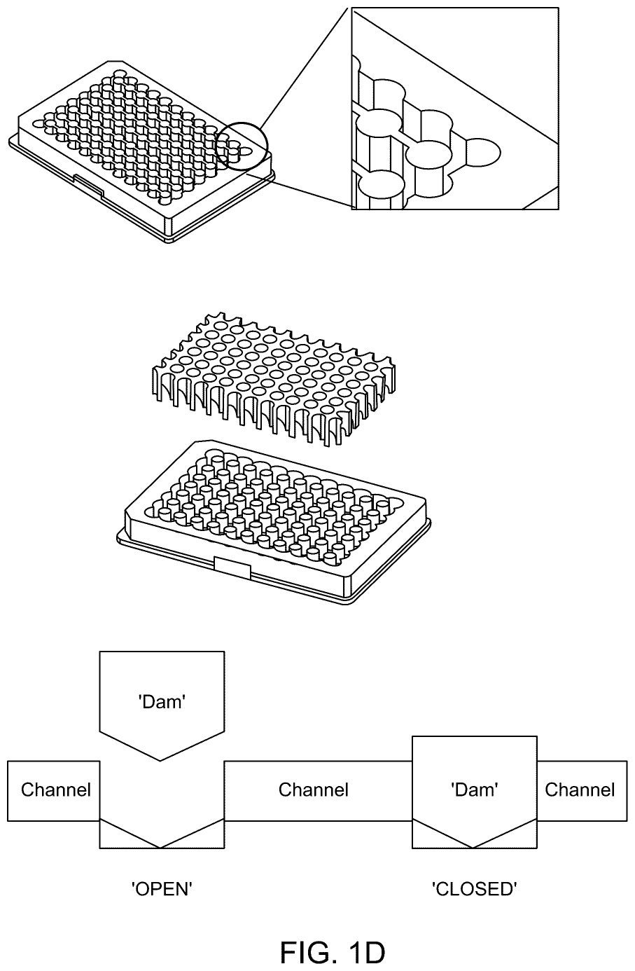

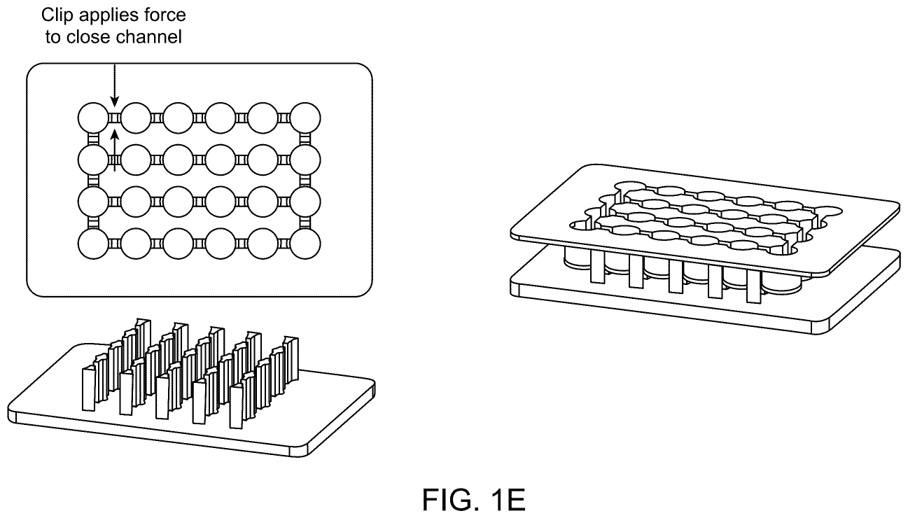

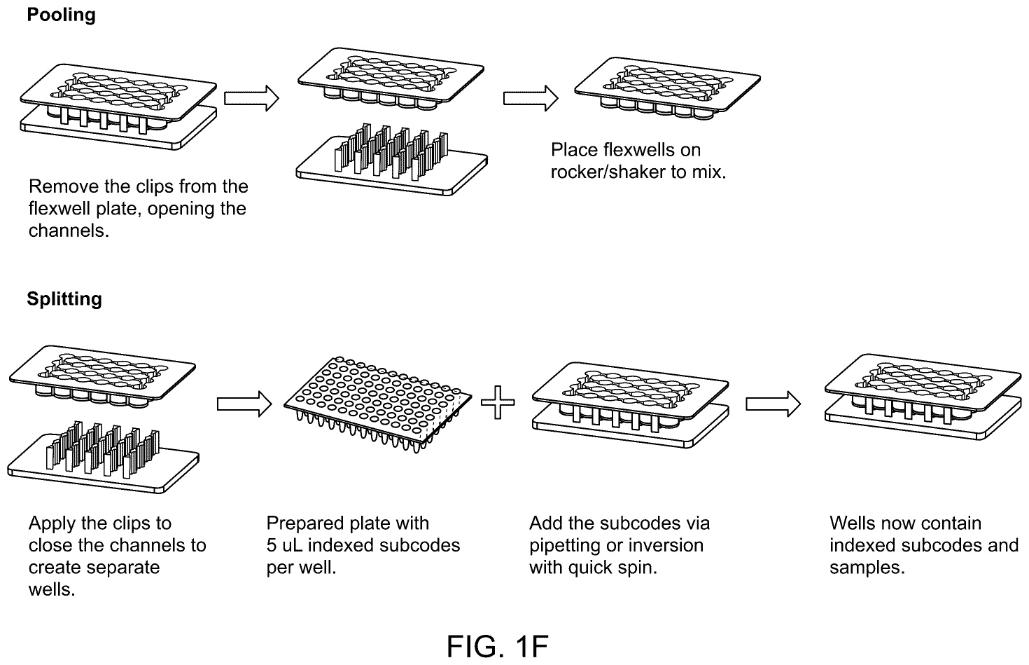

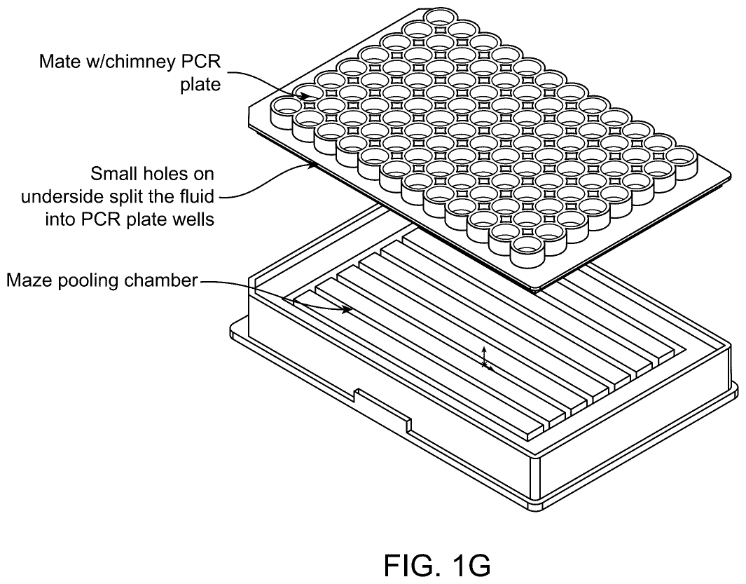

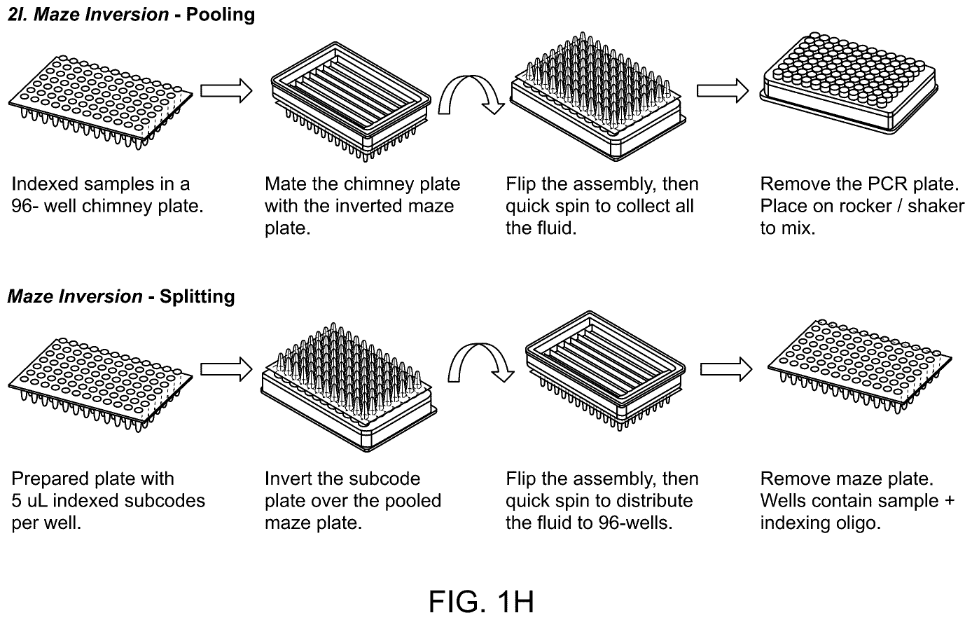

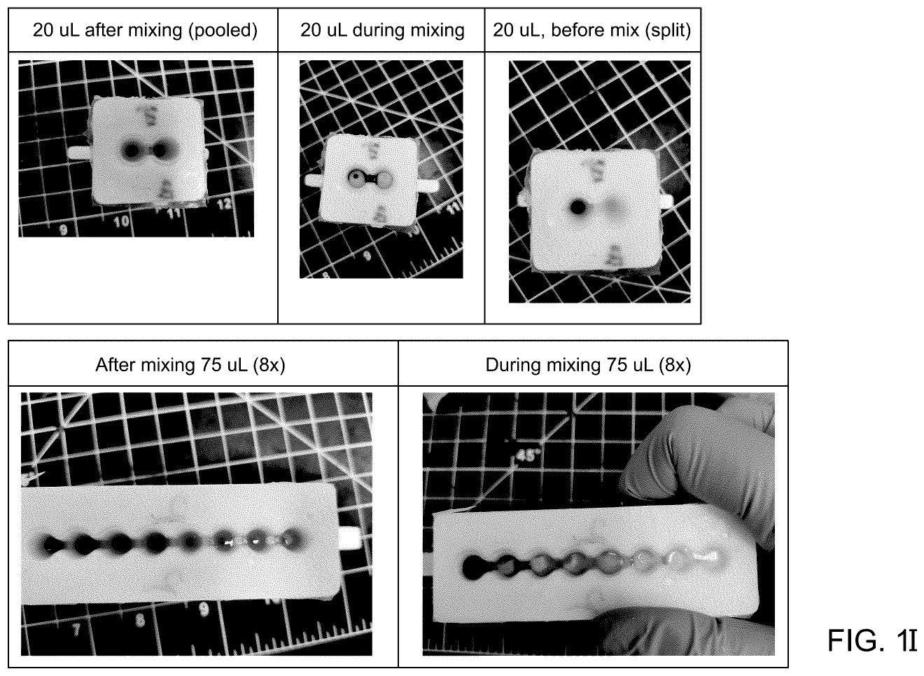

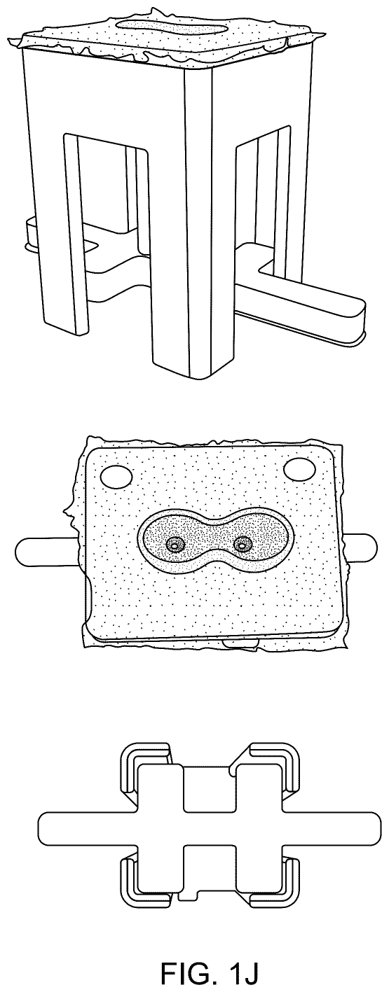

Some implementations of this embodiment of the device are schematically illustrated in FIG. 1A-1D. As illustrated in FIG. 1A, in the multi-compartment plate the compartments may be defined by walls, and the walls may have channels that connect the compartments. In some embodiments, a compartment may be connected to all of the compartments that are adjacent to it on the plate, however, as illustrate in FIG. 1C, this is not necessary. The dam element may contain projections, i.e., pins or small walls, that fit into the channels, for example and the projections and the walls of the channels may have a compatible shape (e.g., a tongue and groove) ensure a junction that minimizes flow between the compartments. The projections could also be made of a flexible material so that a fluid-tight seal is formed when pressure is placed on the dam element. Further examples of the device are illustrated in FIG. 1D. FIG. 1E (left) shows an embodiment of the device that employs a flexible silicone rubber membrane, formed into well shapes (top). In this embodiment, rigid “clips” (bottom) that can be applied to the plate to seal the channels. FIG. 1E (right) shows the assembly, with the clips applied from the bottom. FIG. 1F illustrates how a sample can be split and pooled using the device of FIG. 1E. FIG. 1G shows an example of an inverted dam plate. FIG. 1H illustrates how a sample can be split and pooled using the device of FIG. 1G. FIG. 1I shows results from a dam plate,

In the pooling state, i.e., when the dam element is disengaged from the multi-compartment plate and the channels are open, the liquid inside each compartment can be mixed with the liquid in other compartments by any suitable method, e.g., by rocking, shaking, rotating the plate, which result in mixing of the contents of all of the compartments that are connected. The design can include various number of channels. Channels can connect neighboring wells, diagonal wells, or a combination of the two. Additionally, as illustrated in FIG. 1 C, not all of the compartments need to be connected to all of their adjacent compartments in order for mixing to occur.

As illustrated, in some embodiments the dam element may have openings that are positioned above the compartments of the multi-compartment plate when the multi-compartment plate and dam plate are engaged.

### Stretchable Plate

In some embodiments, the device may comprise (a) a stretchable membrane; and (b) a template comprising openings (e.g., at least 2, least 4, at least 8, at least 16, at least 48, or at least 96 openings). In these embodiments, the device can be switched back and forth between the splitting state and the pooling state by stretching the stretchable membrane through or away from the openings of the template to produce compartments, and then returning the membrane to its original form or close thereto. For example, the membrane can be stretched through the openings or away from the openings without passing through them. In any embodiment, the membrane may be pushed (e.g., by projections or positive pressure) or pulled (e.g., by projections that are attached to a support, or negative pressure). In embodiments in which projections are pushed into the membrane, each compartment may be made using multiple (e.g., 3 or 4 or more) projections, thereby producing a void into which reagents can be delivered. In this embodiment, a stretchable material (e.g. rubber) may be stretched across the surface of a template that has holes in it, and the compartments are made by pushing projections down into the material in the area of the holes. The compartments can be eliminated by removing the projections. The same effect can be applied by a vacuum or other type of pressure.

In any embodiment, the flexible material may overlay the template and may be affixed to the template by, e.g., an adhesive.

In these embodiments, the compartments could be configured as a 48-, 96- or 384-well grid, for example, and in some embodiments, the impressions may be held in place using one or more fasteners. For example, in some cases the projections and template may engage via a snap fit or a press fit, that can be manipulated manually by the user.

In one variation, a pump can create the impressions by applying vacuum that suctions the rubber into a cavity shape (splitting). Removing the vacuum returns it to a single fluid volume (pooling).

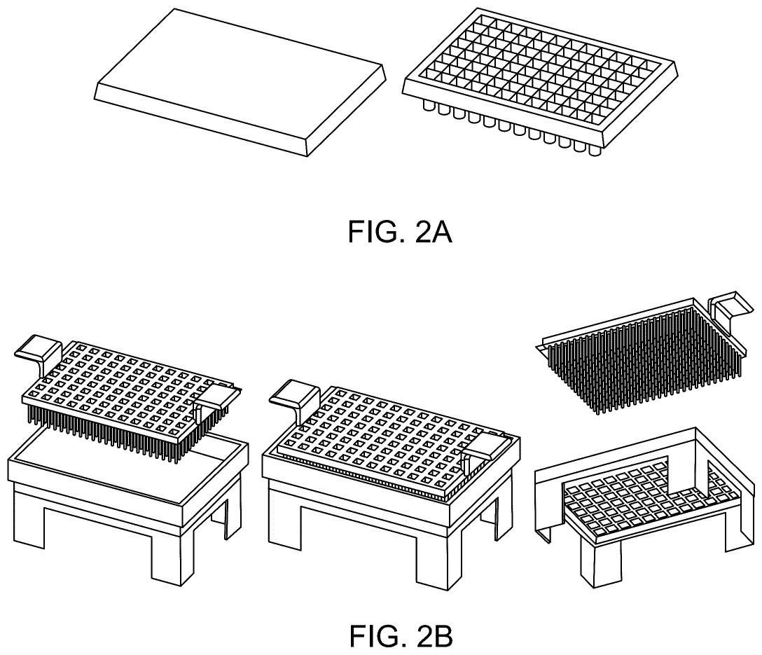

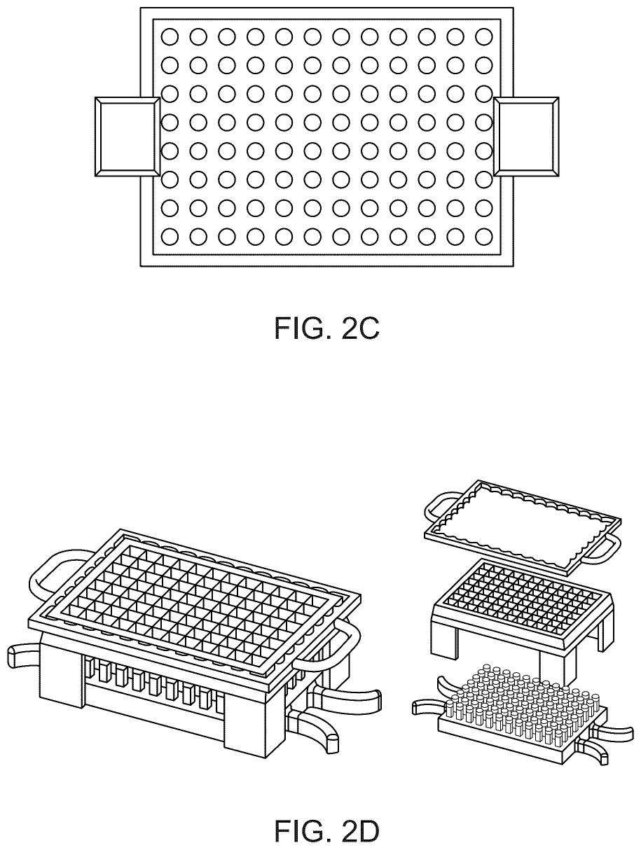

Embodiments of the stretchable membrane implementation of the present device are illustrated in FIGS. 2A-2D. FIG. 2A shows a stretchable membrane in its relaxed state (on the left) and its stretched state (on the right) in which the depressions in the membrane are compartments. In the implementation shown in FIG. 2B, the user moves a grid of projections (extended from a plate) into the stretchable material, pressing into the stretchable material, to place the device in the splitting state in which the compartments are formed. Removal of the projections places the device in the pooling state in which the compartments are removed. FIG. 2B (left) illustrates a device in which the rubber is not stretched, with a single large space for fluid. In this implementation of the device, this state is the pooling state. FIG. 2B (center) illustrates a device in which impressions are generated using projections that are pushed down into the stretchable material from the top. FIG. 2B (right) illustrates the projections, and stretchable membrane mounted onto a template that contains openings. FIG. 2C illustrates a template and membrane from the top.

FIG. 2D illustrates an implementation in which the stretchable membrane that is attached to a grid of projections is pulled downwards from below to create the compartments. The pins may be designed to create a specific well bottom shape like a circle or square, as desired. In this embodiment, the ends of the pins may be bonded to the membrane, e.g., by an adhesive for example.

### Linearly Connected Wells

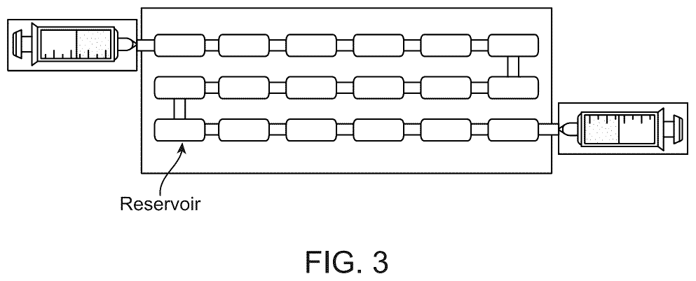

In some embodiments the device may comprise: (a) linearly connected compartments (e.g., at least 2, least 4, at least 8, at least 16, at least 48, or at least 96 compartments) that are closed but connected to one another in series and (b) one or more reservoirs that are fluidically connected to a compartment at the end of the series (i.e., a “terminal” compartment). In these embodiments, the device can be switched back and forth between the splitting state and the pooling state by actuating a pressure differential that forces the sample from the one or more pooling reservoirs into the compartments or that forces the sample the other way, i.e., from the compartments into one or more pooling reservoirs. The pressure differential is generated by a positive pressure or a negative pressure, or a combination of the two. As would be apparent, the pressure differential may be generated by a pump or syringe. In any embodiment, the compartments and one or more pooling reservoirs are in a closed system, operably connected to a one or more syringes or a pump. As illustrated in FIG. 3, the terminal compartments can each be connected to a pressure-driven pump or syringe, thereby providing a way to propel the sample from the reservoirs to the compartments and back again. In these embodiments, the compartments could have pre-spotted indexed oligonucleotides that are reconstituted when the sample is added.

### Physiochemical Approaches.

Switching between splitting and pooling states can be controlled by several physiochemical approaches including modulating surface hydrophobicity on channels or surfaces connecting regions or wells. Exemplar approaches include modulating surface hydrophobicity by optical or electrical control such as in optoelectrical and electrical wetting methods. (e.g. Advanced Liquid Logic, SONY CCD, Berkeley Lights, etc.). Jia Li and Chang-Jin Kim Current commercialization status of electrowetting-on-dielectric (EWOD) digital microfluidics Lab on a Chip 2020, 20, 1705-1712 and references cited herein. Cellular samples or particles can be pooled and split onto such devices through the use of an optical or electrical stimulus. Especially systems that have open access and enable the additional of barcodes or indices to specific compartments are desirable. A non-limiting example is Zichuan Yi et al. Design of an Open Electrowetting on Dielectric Device Based on Printed Circuit Board by Using a Parafilm M, Front. Phys., 2020|https://doi.org/10.3389/fphy.2020.00193. Systems can interface with liquid handling systems or micro-dispense systems to add barcodes, reagents, enzymes etc to specific regions or (temporary) compartments during the split-pool process.

### Funnel Plate

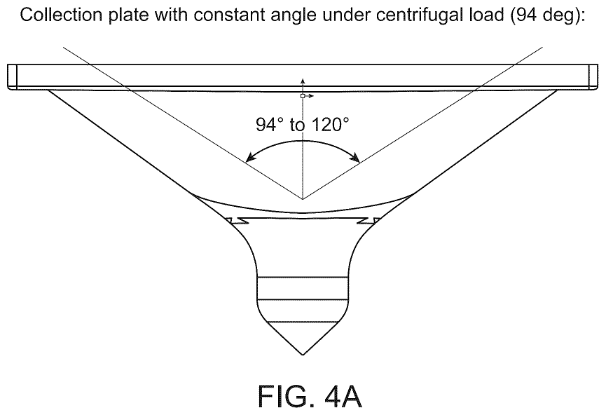

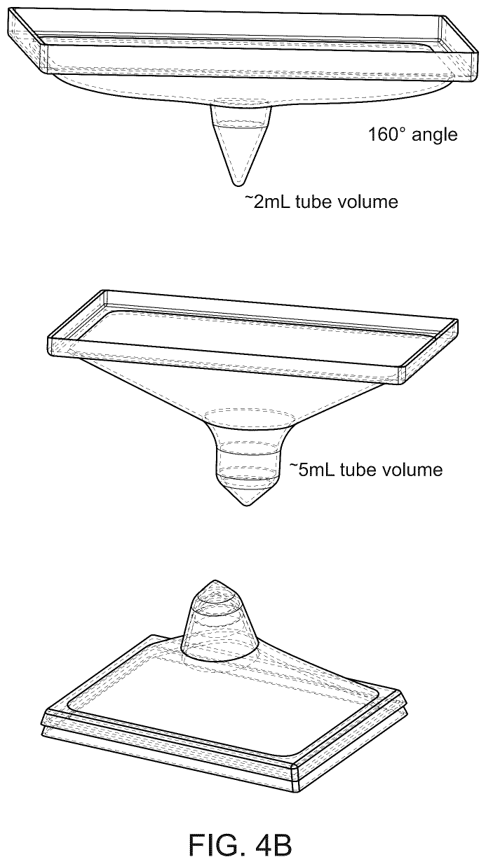

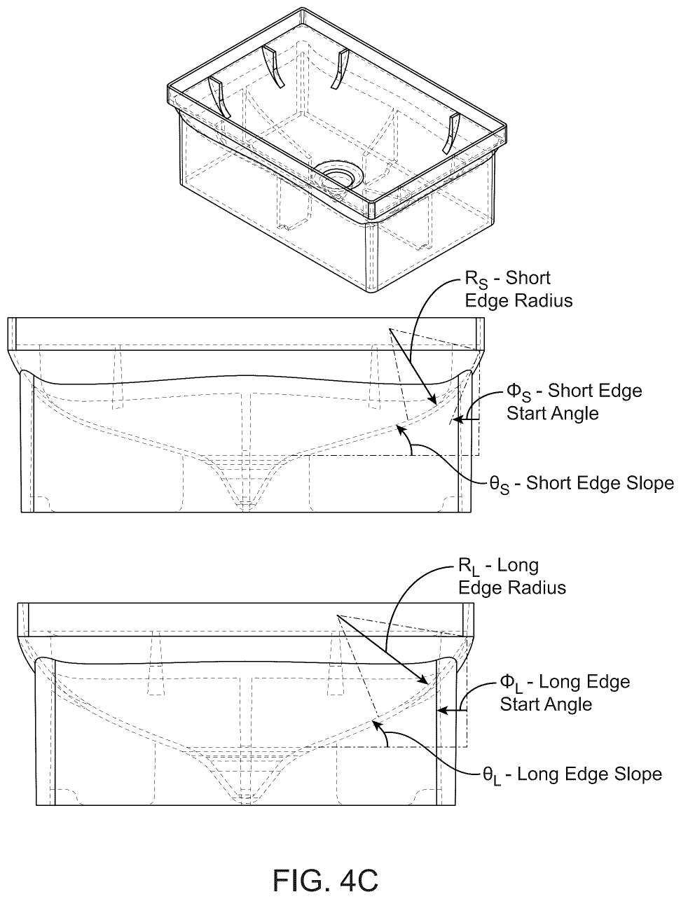

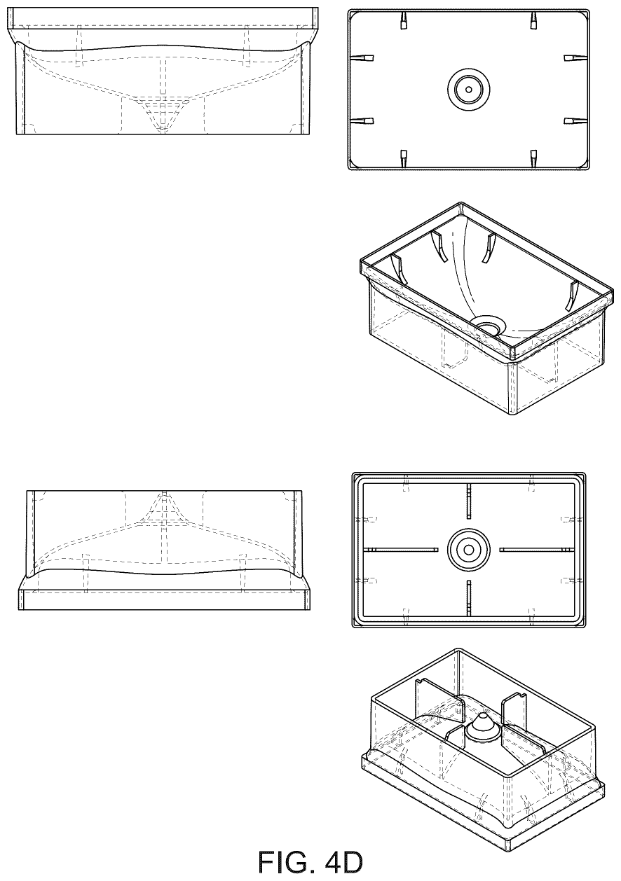

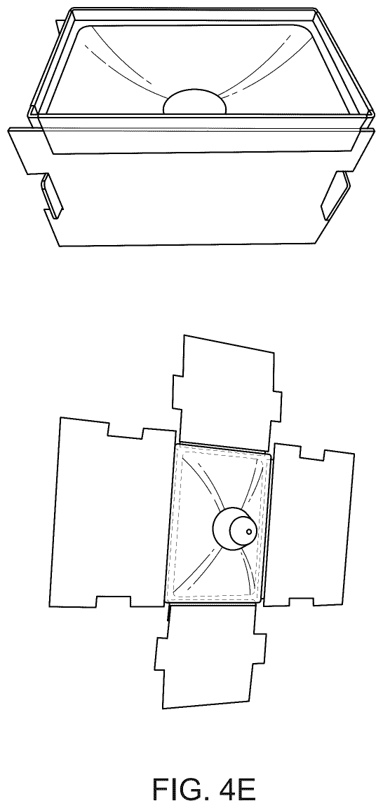

A device for collecting a sample or samples from a multi-well plate is also provided (FIG. 4A-B). In these embodiments, the device may comprise a lip that is adapted to mate with the multi-well plate, a funnel, wherein the wall-to-wall angle of the funnel can be any angle e.g. in the approximate range of 91° to 17°, and a cylindrical reservoir at the bottom of the funnel, distal to the lip, ending in a V- or U-shaped bottom, suitable for inspecting a pellet of cells or nuclei and which, in some embodiments, may be made of a transparent plastic, e.g., single piece of molded plastic (FIG. 4A). In some embodiments, the bottom of the funnel is used to collect cells or Nuclei and wash them. The volume of the V- or U-shaped bottom may be in the range of 0.5 mL to 15 mL.

This device may be used for collecting a sample or samples from a multi-well plate, where the method may comprise mating the device with a multi-well plate (i.e., placing the lip over the top of the plate), and applying a centrifugal force to push the sample from the plate into reservoir via the funnel, thereby collecting the sample in the reservoir. The centrifugal force may be in the range of 50 g, 500 g, or 5000 g or higher and the spin time will vary based on both the material being collected and the plate it is being collected from. For example, one embodiment of these spin settings might be 300 g for 5 min for cells in a 96-microwell plate. In some embodiments the interior of the funnel may be highly polished or coated with a low-binding material to reduce cell or nuclei adhering to the surface of the funnel (FIG. 4B). In some embodiments, the funnel may include an integrated skirt feature to fit the funnel and plate in a centrifuge. In some embodiments, an adapter is used to fit the funnel plate in a centrifuge. In some embodiments, these plates may be stackable (FIG. 4C). In some embodiments, the funnel contains legs, a skirt, or an adapter that is attached to the funnel and can be folded to support standing (FIG. 4D). In some embodiments, the funnel may have the general shape and dimensions similar to standard laboratory plastics (e.g. multi-well plate dimensions), and fit a wide-range of common laboratory instruments (e.g. centrifuge, temperature control devices etc). Common dimensions are SBS Footprint, Stands for “Society for Biomolecular Screening”. This form factor is also referred to as SLAS/ANSI form factor.

In some embodiments and with reference to FIGS. 4A-D, the wall that defines a funnel may be composed of a radius and slope on both the long and short edges. The slope angle can be any angle from 5° to 35°, relative to the horizontal. In any embodiment, the radius of the profile can be any radius from 25 mm to 50 mm. the radius start angle, relative to the vertical, can be any angle from 0° to 25°, for example. In some embodiments, the V- or U-shaped bottom is highly polished (i.e., smooth, not etched or rough) on both the interior and exterior to allow clear inspection of any sample in that reservoir.

### Dimensions and Materials

Any of the devices described herein may have dimensions that are the same as a standard microtiter plate, which has length of approximately 127 mm, a width of approximately 85 mm and the height of approximately 14 mm. The funnel plate, because it has a funnel region, may contain a “base” that is roughly the size of a multi-well plate. The funnel may be several cm in length, meaning that the funnel may have a height of about 50 mm. In some cases, a device may contain 96 containers, arranged 8/12, centered in 9 mm intervals, or 24, 48, or 384 containers, arranged accordingly. In general terms, the surface that makes contact with the sample should be made of a material that allows the sample to be recovered at a high efficiency. For example, silicone rubber or another material which has the properties of chemical inertness, low binding surface, temperature resistance, and hydrophobic surface energy, and high elasticity could be used as the stretchable material. In many embodiments, a hydrophobic surface can be used. In some cases, high hydrophilicity may be desired, to ensure spreading of fluid across features. Either hydrophobicity or hydrophilicity can improve cell or nuclei retention “low binding” due to low molecular adsorption characteristics.

The methods and compositions discussed in this application can be used in combination with or are part of the process outlined in the following patents: Nolan et al. U.S. Pat. Nos. 10,144,950, 10,174,310, 10,626,442, Ser. No. 16/114,250 and are incorporated in its entirety herein.

### EXAMPLES

To further illustrate some embodiments of the present invention, the following specific examples are given with the understanding that they are being offered to illustrate examples of the present invention and should not be construed in any way as limiting its scope.

### Example 1

### Collection of Cells/Nuclei Using Funnel Plate

In this example, cell and nuclei sample collection is demonstrated using a a funnel plate. Nuclei are isolated or cells are prepared, then distributed at a specified concentration into a standard well-plate. The funnel plate is then placed on top of the well-plate and their mating features are aligned. The pair is then flipped so that the openings of the well-plate are facing downward, into the cylindrical reservoir at the bottom of the funnel. The pair is placed into a centrifuge bucket (with an appropriate balance in the opposing bucket) and spun. The collected material is then resuspended and transferred from the funnel plate to a tube for quantification. In this example, a 96-well plate was used with 25,000 cells or nuclei per well. The funnel plate/well-plate pair was spun at 300 g for 5-1 min. Testing was done on an Eppendorf 5920R centrifuge with a S-4x1000 rotor (portrait format) and a ThermoFisher Sorvall Legend XTR centrifuge with a TX-1000 Rotor (landscape format). The cells used was Jurkat, Clone E6-1. Cellular samples were recovered using the funnel with 90.7%, and nuclei samples can be collected with 89.0% recovery.

Detailed description of the protocol using cells or nuclei:
1. Weigh both plates before proceeding so initial volume can be determined.
2. Distribute cell or nuclei to wells on a plate, loading 25,000 cell or nuclei per well in 15 uls of APB.
   a. First add the nuclei to 8 or 12 wells of strip tubes.
   b. Then using a multi-channel pipettor, add the nuclei to the wells on a plate.
3. Seal the plates with plastic adhesive seal.
4. Briefly centrifuge the plates to collect fluid at the bottom.
5. Unseal the plates.
6. Weigh well plates and use empty weight to determine fluid volume in plate:

$${V}_{fluid} = ( {m}_{filled} - {m}_{empty} ) \cdot 1000$$

Where V_{fluid} is in μL and m_{filled} and m_{empty} are in grams
7. Put the funnel on top of the 96 well plate. Re-seal and reserve the second on ice for pipetting during centrifuge spin or after funnel collecting is complete.
8. Quickly flip the plate/funnel assembly.
9. Weight and balance with appropriate balancing plate.
10. Centrifuge in swing bracket rotor @ 300 g for 3-5 min at 4° C. The exact spin time will depend on the material being spun. We had good performance with these settings while testing with a mixture of water and 0.05% methylene blue. Begin with the higher time and decrease as needed to prevent pelleting.
11. Perform thorough resuspension on the collected volume. This prevents cells which have been driven onto the walls of the funnel from being lost in collection.
12. Measure collected volume.
   a. Using a pipette pull exactly the quantity of fluid from the funnel. This will probably take two pipetting actions: One for 1 mL and one for the remainder. The remainder should be taken such that all fluid is collected with no bubbles.
   b. Deposit it into a 1.5 mL tube
13. Take 10 uL of the cell/nuclei and mix with 10 uL of 0.4% trypan blue.
14. Quant on Countess, Luna, TC20 or a hemocytometer.

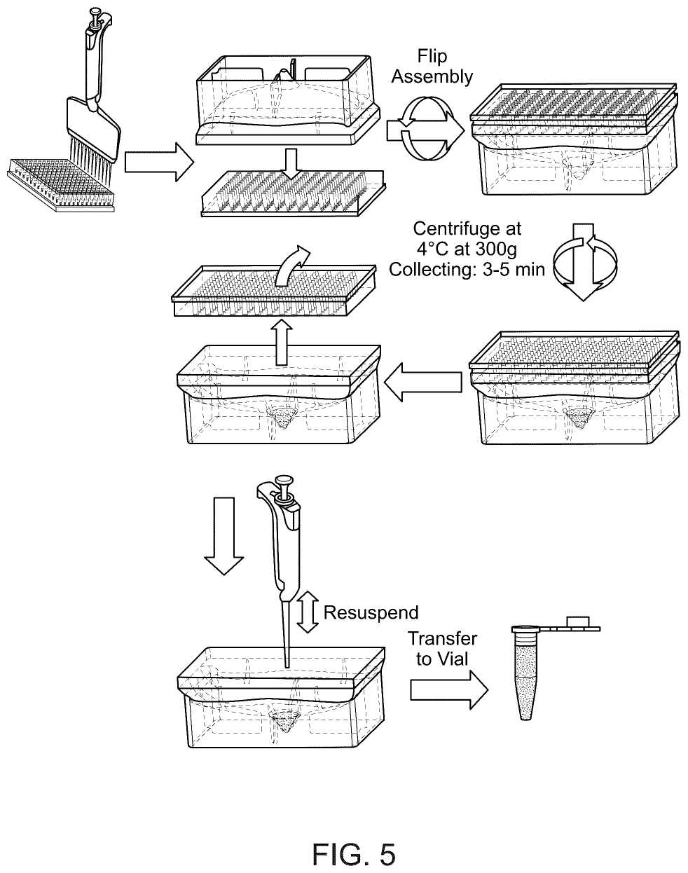

The approach used for these experiments is illustrated in FIG. 5. A summary of the results is shown in the table below.

| | Sample | | Centrifuge | Initial Conc. | Recovery Time |
|---|---|---|---|---|---|
| Sample | Used: | Test Type | Format | (Cells/mL) | (min, sec) |
| 1 | Jurkat | Collecting | Portrait | 1.19 | 300 g, 5 min |
| 2 | Jurkat | Collecting | Portrait | 1.56 | 300 g, 5 min |
| 3 | Jurkat | Collecting | Portrait | 1.79 | 300 g, 5 min |
| 4 | Jurkat | Collecting | Portrait | 1.79 | 300 g, 5 min |
| 5 | Jurkat | Collecting | Portrait | 1.78 | 300 g, 5 min |
| 6 | Jurkat | Collecting | Portrait | 1.78 | 300 g, 5 min |
| 7 | Jurkat | Collecting | Landscape | 0.66 | 300 g, 5 min |
| 8 | Jurkat | Collecting | Landscape | 1.32 | 300 g, 5 min |
| 9 | Jurkat | Collecting | Portrait | 1.83 | 300 g, 1 min |
| 10 | Jurkat | Collecting | Portrait | 1.48 | 300 g, 1 min |
| 11 | Jurkat | Collecting | Portrait | 1.72 | 300 g, 1 min |

| Initial | | | | | % cells after all |
|---|---|---|---|---|---|
| Plate | Recovered | Average | | % volume | steps (Does not |
| Volume | Volume | Conc. (e6 | Viability | after all | account for |
| (μL) | (μL) | Cells/mL) | (%) | steps | viability) |
| 1510 | 1440 | 1.32 | | 95.40% | 106% |
| 1420 | 1400 | 1.51 | | 99% | 95% |
| 1430 | 1379 | 1.985 | 23.5 | 96.40% | 107% |
| 1370 | 1355 | 1.745 | 14 | 98.90% | 96% |
| 1400 | 1337 | 1.19 | 95.5 | 95.50% | 64% |
| 1374 | 1351 | 1.26 | 95 | 98.30% | 70% |
| 1410 | 1310 | 0.721 | | 92.90% | 101% |
| 310 | 280 | 1.1 | | 90.30% | 75% |
| 1395 | 1317 | 1.365 | 91.5 | 94.40% | 70% |
| 1396 | 1336 | 1.325 | 95.5 | 95.70% | 86% |
| 1388 | 1348 | 0.385 | 81.5 | 97.10% | 22% |
| | | Cells | Collecting | 96.50% | 90.70% |
| | | Nuclei | Collecting | 99.80% | 89.00% |

### Example 2

### Pelleting of Cells/Nuclei Using Funnel Plate

In this example, cell sample pelleting is demonstrated using the novel funnel. Cells are prepared, then distributed into a standard well-plate of arbitrary density. The funnel is then placed on top of the 96 well-plate and their mating features are aligned. The pair is then flipped so that the openings of the well-plate are facing downward, into the cylindrical reservoir at the bottom of the funnel. The pair is placed into a centrifuge bucket (with an appropriate balance in the opposing bucket) and centrifuged. The supernatant is removed and placed in a tube for quantification. The pellet is then resuspended and transferred from the funnel to a tube for quantification. In this example 96-wells plates were used with 25,000 cells or nuclei per well. The funnel/well-plate pair was spun at 300 g for 10 min. Testing was done on an Eppendorf 5920R centrifuge with a S-4x1000 rotor (portrait format). The cells used in this testing was Jurkat, Clone E6-1. This protocol has demonstrated that, using the funnel, cellular samples can be pelleted with 78.1% recovery.

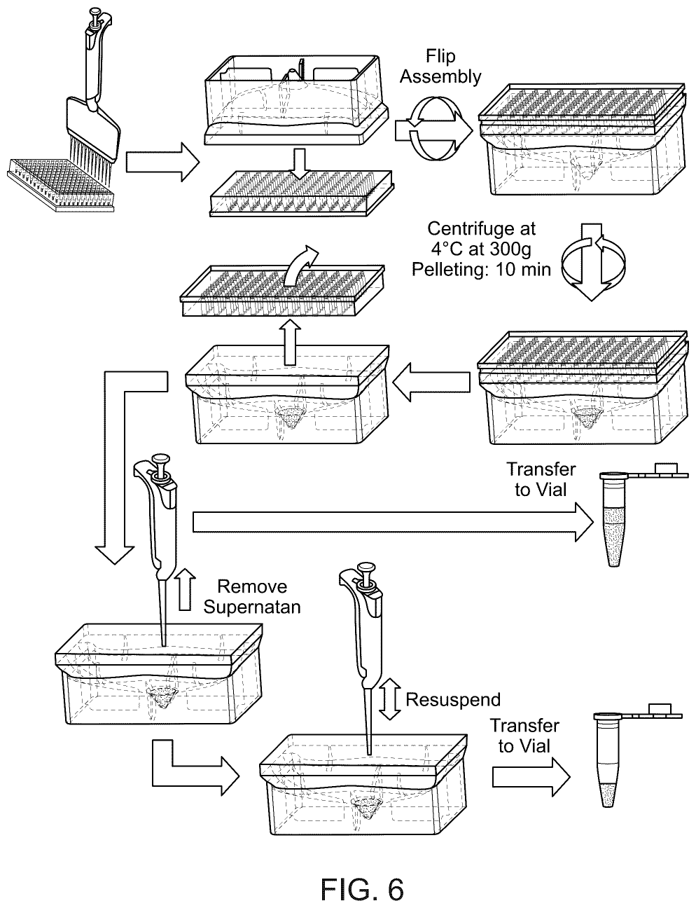

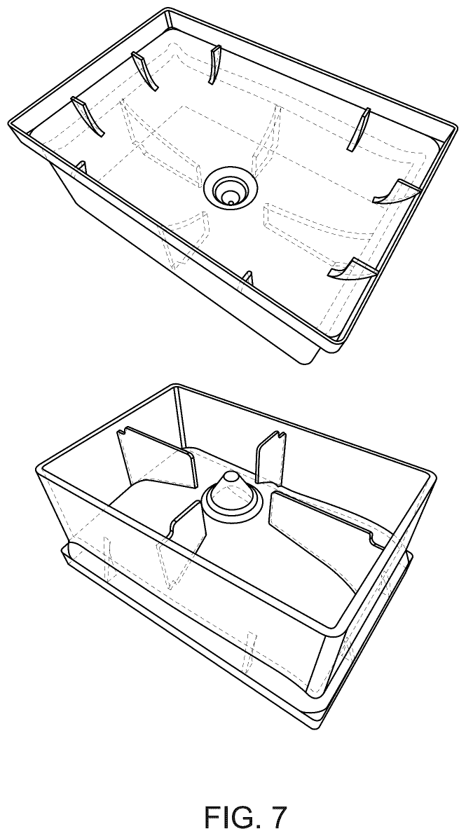

The protocol used is described below and can be completed with cells or nuclei:
1. Weigh both plates before proceeding so initial volume can be determined.
2. Distribute cells/nuclei to wells on a plate. Plan the experiment for loading 25,000 cells/nuclei per well in 15 μL of APB.
   a. First add the nuclei to 8 or 12 wells of strip tubes.
   b. Then using a multi-channel pipettor, add the nuclei to the wells on a plate.
3. Seal the plates with plastic adhesive seal.
4. Briefly centrifuge the plates to collect fluid at the bottom.
5. Unseal the plates Weigh well plates and use empty weight to determine fluid volume in plate:

$${V}_{fluid} = ( {m}_{filled} - {m}_{empty} ) \cdot 1000$$

Where V_{fluid} is in μL and m_{filled} and m_{empty} are in grams
6. Put the funnel on top of one plate. Re-seal and reserve the second on ice for pipetting during centrifuge spin or after funnel collecting is complete.
7. Weight and balance with appropriate balancing plate.
8. Centrifuge in swing bracket rotor @ 300 g for 10 min at 4° C.
9. Remove the plate/funnel assembly from the centrifuge. Remove and discard the plate.
10. Carefully aspirate the majority of supernatant leaving 25 μL behind. Since you might not see the pellet do not go all the way to the bottom of the funnel.
11. Keep supernatant for future nuclei quant.
12. Resuspend pellet (might be invisible) using 200 μl wide bore tip.
13. Take 10 uL of the nuclei and mix with 10 μL of 0.4% trypan blue.
14. Make a second sample for quant using the supernatant from the previous step (10 μL of supernatant mixed with 10 μL of 0.4% trypan blue solution).
15. Quant on Countess, Luna, TC20 or a hemocytometer.
   The workflow for the experiments is illustrated in FIG. 6. A summary of the results are shown in the table below.

| | Sample | | Centrifuge | Initial Conc. | |
|---|---|---|---|---|---|
| Sample | Used: | Test Type | Format | (Cells/mL) | Recovery Time (min, sec) |
| 1 | Jurkat | Pelleting | Portrait | 1.07 | 300 g, 10 min |
| 2 | Jurkat | Pelleting | Portrait | 1.21 | 300 g, 10 min |

| Initial Plate | Recovered | Average | | | |
|---|---|---|---|---|---|
| Volume | Volume | Conc. (e6 | Viability | % volume | % cells after all steps (Does |
| (μL) | (μL) | Cells/mL) | (%) | after all steps | not account for viability) |
| 1450 | 1450 | 0.953 | | 100% | 89% |
| 1260 | 1400 | 0.731 | | 111% | 67% |
| | | Cells | Pelleting | 106% | 78.10% |

### Example 3

### Design Iteration of Funnel

In this example, the design of the funnel was iterated to understand the features which drive an effective design and shorten the overall height to increase compatibility with a large variety and commonly used centrifuges. Testing was done using cell buffer (PBS, 2% BSA) with 0.01% methylene blue to enhance visibility, using the collected or trapped fluid as an initial indicator of performance. When a design was a baseline, or sufficiently developed, cell collecting and pelleting were also run using the techniques described in Examples 1 and 2, above. In cases where cells or nuclei are not used, the blue dyed buffer is prepared, then distributed at volume matching that seen in cell or nuclei testing into a standard 96 well-plate. The funnel and 96-well plate is centrifuged as in the collecting procedure and the collected volume is quantified. Any volume undesirably left in the corners of the plate is also quantified. In this example 96-wells plates were used with 15 uL per well. The funnel plate/well-plate pair was spun at 300 g for 5 min. Testing was done on an Eppendorf 5920R centrifuge with a S-4x1000 rotor (portrait format), a ThermoFisher Sorvall™ Legend XTR centrifuge with a TX-1000 Rotor (landscape format), and a ThermoFisher Sorvall™ ST 8 centrifuge with a M-10 Rotor (landscape format). When cells were used, they were Jurkat, Clone E6-1, at 25,000 cells per well. The testing demonstrated that:
1. CNC parts perform similarly to injection molded parts at volume and cell collection
2. Additively manufactured PLA parts which were epoxy coated perform similarly to injection molded parts at volume collection
3. The addition of a short edge radius to the slope profile (R_{S} in FIG. 4A) is essential to functionality in some landscape centrifuges, and that this radius has more of a role in functionality than the short edge slope (θ_{S} in FIG. 4A)
4. Supporting the well-plate with a minimum surface area and reducing the number of tight radii which the plate contacts prevents fluid from being left behind and increases collected volume.

The protocol used for fluid functionality testing is described below. For cell or nuclei collecting see examples 1 and 2:
1. Weigh well plate before proceeding so fluid volume before centrifuge spinning can be determined
2. Distribute 15 μL of dH2O and 0.05% methylene blue to each well on a plate.
   a. First add the dH2O mixture to 8 or 12 wells of strip tubes, including 15 μL more than needed to fill the wells to accommodate loss.
   b. Then using a multi-channel pipettor, add the nuclei to the wells on a plate.
3. Seal the plate with plastic adhesive seal.
4. Briefly centrifuge the plate to collect fluid at the bottom.
5. Unseal the plate.
6. Weigh well plate and use empty weight to determine fluid volume in plate:

$${V}_{fluid} = ( {m}_{filled} - {m}_{empty} ) \cdot 1000$$

Where V_{fluid} is in μL and m_{filled} and m_{empty} are in grams
7. Put the funnel on top of the plate.
8. Quickly flip the plate/funnel assembly by holding firm on plate and funnel with both hands.
9. Place plate and funnel in centrifuge
10. Add balancing plate with appropriate counter-weight, i.e. same weight as plate and funnel.
11. Centrifuge in swing bracket rotor @ 300 g for 5 min at 4° C.
12. Measure and record the collected volume
   a. Using a pipette pull exactly the quantity of fluid from the funnel. This will probably take two pipetting actions: One for 1 mL and one for the remainder. The remainder should be taken such that all fluid is collected with no bubbles.
   b. Deposit it into a 1.5 mL vial
      The protocol and workflow used for the experiments is illustrated in FIG. 5. A summary of the of the results are shown in the tables below.

| Test no | Funnel Name | Manufacturing Method |
|---|---|---|
| 1 | Version 1 | Injection Molding |
| 2 | Version 1 | CNC Machined |
| 3 | Version 1 | Epoxy coated Injection Mold |
| 4 | Version 1 | Epoxy Coated PLA |
| 5 | Shortened Version 1 | Injection Molding, Epoxy Coated PLA |
| 6 | Version 3-1.0 | CNC Machined |
| 7 | Version 3-1.01 | Epoxy Coated PLA |
| 8 | Version 3-1.1 | Epoxy Coated PLA |
| 9 | Version 3-1.2 | Epoxy Coated PLA |
| 10 | Version 3-1.3 | Epoxy Coated PLA |
| 11 | Version 3-1.4 | Epoxy Coated PLA |
| 12 | Version 3-1.5 | Epoxy Coated PLA |
| 13 | Version 3-1.5 | Epoxy Coated PLA |
| 14 | Version 3-1.5 | Epoxy Coated PLA |
| 15 | Version 3-1.5 | Epoxy Coated PLA |
| 16 | Version 3-1.5.1 | Epoxy Coated PLA |
| 17 | Version 3-1.5.1 | Epoxy Coated PLA |
| 18 | Version 3-1.5.1 | CNC Machined |
| 19 | Version 4 | Injection Molding |
| 20 | Version 4 | Injection Molding |
| 21 | Version 4 | Injection Molding |

| | | | Short Edge | Long Edge | Conical | |
|---|---|---|---|---|---|---|
| Test | Short Edge | Long Edge | Bowl Radius | Bowl Radius | Volume | Height |
| no. | Slope (deg) | Slope (deg) | (mm) | (mm) | (mL) | (mm) |
| 1 | 31.52 | 28.52 | 0 | 42.17 | 11 | 72.95 |
| 2 | 31.52 | 28.52 | 0 | 42.17 | 11 | 72.95 |
| 3 | 31.52 | 28.52 | 0 | 42.17 | 11 | 72.95 |
| 4 | 31.52 | 28.52 | 0 | 42.17 | 11 | 72.95 |
| 5 | 31.52 | 28.52 | 0 | 42.17 | 11 | 56.12 |
| 6 | 30 | 30 | 0 | 22.85 | 1 | 56.74 |
| 7 | 31.52 | 28.52 | 0 | 42.17 | 1 | 58.44 |
| 8 | 16 | 28.52 | 29.67 | 42.17 | 1 | 58.93 |
| 9 | 28.52 | 28.52 | 9.87 | 42.17 | 1 | 58.93 |
| 10 | 5 | 28.52 | 36.5 | 42.17 | 1 | 58.93 |
| 11 | 5 | 35 | 47.58 | 49.36 | 1 | 62.39 |
| 12 | 16 | 28.52 | 33.96 | 45.43 | 1 | 59.3 |
| 13 | 16 | 28.52 | 33.96 | 45.43 | 1 | 59.3 |
| 14 | 16 | 28.52 | 33.96 | 45.43 | 1 | 59.3 |
| 15 | 16 | 28.52 | 33.96 | 45.43 | 1 | 59.3 |
| 16 | 16 | 28.52 | 33.96 | 45.43 | 1 | 58.9 |
| 17 | 16 | 28.52 | 33.96 | 45.43 | 1 | 58.9 |
| 18 | 16 | 28.52 | 33.96 | 45.43 | 1 | 58.9 |
| 19 | 16 | 28.52 | 33.96 | 45.43 | 1 | 58.9 |
| 20 | 16 | 28.52 | 33.96 | 45.43 | 1 | 58.9 |
| 21 | 16 | 28.52 | 33.96 | 45.43 | 1 | 58.9 |

| | Type: Portrait | | | | | | | | |
|---|---|---|---|---|---|---|---|---|---|
| | Centrifuge: Eppendorf 5920R | | | Type: Landscape | | | Type: Landscape | | |
| | Rotor: S-4x1000 | | | Centrifuge: ThermoFisher | | | Centrifuge: ThermoFisher | | |
| | | % | | Sorvall ™ ST 8 | | | Sorvall ™ Legend XTR | | |
| | | Volume | | Rotor: M-10 | | | Rotor: TX-1000 | | |
| Test | % | caught in | | | % Vol in | | % | % Vol in | |
| no | Volume | corners | % Cell | % Vol | corners | % Cell | Volume | corners | % Cell |
| 1 | 96.86% | 1.39% | 75.55% | — | — | — | 99.00% | 0.00% | — |
| 2 | 94.56% | 0.00% | 78.48% | — | — | — | — | — | — |
| 3 | 96.67% | 0.00% | — | — | — | — | — | — | — |
| 4 | 96.87% | 0.00% | — | — | — | — | 95.00% | 0.00% | — |
| 5 | 100.00% | 0.00% | — | 35.96% | 15.84% | — | — | — | — |
| 6 | 93.96% | 0.00% | 100.06% | — | — | — | 97.50% | 2.50% | — |
| 7 | 95.83% | 0.00% | — | 34.56% | 23.83% | — | — | — | — |
| 8 | 95.89% | 0.00% | — | 86.82% | 0.03% | — | — | — | — |
| 9 | 95.30% | 0.00% | — | 30.20% | 30.87% | — | — | — | — |
| 10 | 97.30% | 2.36% | — | 83.30% | 3.21% | — | — | — | — |
| 11 | — | — | — | 88.51% | 0.00% | — | — | — | — |
| 12 | — | — | — | 86.49% | 0.00% | — | — | — | — |
| 13 | — | — | — | 83.78% | 0.00% | — | — | — | — |
| 14 | — | — | — | 89.26% | 0.00% | — | — | — | — |
| 15 | 95.96% | 0.00% | 96.53% | — | — | — | — | — | — |
| 16 | — | — | — | 87.14% | 0.00% | 12.99% | — | — | — |
| 17 | 97.26% | 0.00% | 51.87% | 89.04% | 0.00% | 31.00% | 93.15% | 0.00% | 57.01% |
| 18 | 94.89% | 0.00% | 80.33% | 82.76% | 0.00% | 47.32% | — | — | — |
| 19 | 97.20% | 0.00% | — | 87.25% | 2.68% | — | — | — | — |
| 20 | 96.47% | 0.00% | 90.67% | 94.83% | — | 84.29% | — | — | — |
| 21 | 105.56% | — | 78.10% | 93.62% | — | 85.70% | — | — | — |

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

That which is claimed is:

1. A device for splitting and pooling a biological sample, wherein the device is capable of being switched back and forth between:
(i) a splitting state in the which a pooled sample becomes split into multiple compartments; and
(ii) a pooling state in which a sample that is split between multiple compartments in (i) becomes pooled.

2. The device of claim 1, wherein in splitting state the pooled sample becomes split into at least 2, least 4, at least 8, at least 16, at least 48, or at least 96 compartments.

3. The device of claim 1 or 2, wherein the compartment has a volume in the range of 5 ul to 50 ml.

4. The device of any of claims 1-3, wherein the device comprises:
(a) a multi-compartment plate in which the compartments are fluidically connected by channels; and
(b) a dam element that is adapted to engage with the multi-compartment plate and block at least some of the channels;
wherein the device can be switched back and forth between the splitting state and the pooling state by engaging and disengaging the dam element with/from the multi-compartment plate.

5. The device of claim 4, wherein the multi-compartment plate comprises a flexible material and wherein the flexible material blocks the channels by being pushed or squeezed into the channel.

6. The device of claim 4, wherein dam element comprises projections, and wherein engaging the dam element with the multi-compartment plate pushes the projections into the channels to block the channels.

7. The device of claim 6, wherein the dam element has openings that are positioned above the compartments of the multi-compartment plate when the multi-compartment plate and dam plate are engaged.

8. The device of any of claims 1-3, wherein the device comprises
(a) a stretchable membrane; and
(b) a template comprising openings;
wherein the device can be switched back and forth between the splitting state and the pooling state by stretching the stretchable membrane through or away from the openings of the template to produce compartments, and then returning the membrane to its original form.

9. The device of claim 8, wherein the membrane is pushed through or away from the openings of the template.

10. The device of claim 8, wherein the membrane is pulled through or away from the openings of the template.

11. The device of any of claims 8-10, wherein the membrane is stretched using projections, positive pressure or negative pressure.

12. The device of any of claims 8-11, wherein the membrane is stretched using projections, and multiple projections produce each compartment.

13. The device of any of claims 8-12, wherein the membrane is stretched using projections, and the ends of the projections are affixed to the membrane.

14. The device of any of claims 1-3, wherein the device comprises:
(a) compartments that are fluidically connected in series; and
(b) one or more pooling reservoirs that are fluidically connected to a terminal compartment,
wherein the device can be switched back and forth between the splitting state and the pooling state by actuating a pressure differential that forces the sample from the one or more pooling reservoirs into the compartments or that forces the sample from the compartments into one or more pooling reservoirs.

15. The device of claim 14, wherein the pressure differential is generated by a positive pressure.

16. The device of claim 14, wherein the pressure differential is generated by a negative pressure.

17. The device of any of claims 14-16, wherein the pressure differential is generated by a pump or syringe.

18. The device of any of claims 14-17, wherein the compartments and one or more pooling reservoirs are in a closed system, operably connected to a one or more syringes or pumps.

19. The device of any of claims 14-17, wherein the device comprises (a) compartments that are fluidically connected in series and syringes that are connected to the terminal compartments.

20. A method for indexing a sample using a split and pool approach, comprising, in order:
(a) adding subunits of an index to a split sample that is present in the device of any prior claim, in its splitting state; and
(b) switching the device to the pooling state, thereby pooling the split sample of step (a).

21. The method of claim 20, wherein the split sample of (a) is made by:
loading one more samples onto the device when the device is in the splitting state.

22. The method of claim 20, wherein the split sample of (a) is made by:
switching the device from a pooling state to a splitting state, thereby splitting a pooled sample into multiple compartments.

23. The method of claim 20, further comprising:
(c) allowing the pooled sample produced in (c) to mix;
(d) switching the device to a splitting state, thereby splitting the pooled sample into multiple compartments; and
(e) repeating steps (a) and (b) at least once, each repeat followed by steps (c) and (d), optionally except for the final repeat, to produce an indexed sample.

24. The method of claim 23, further comprising:
(f) collecting the indexed sample after step (e).

25. The method of any of claims 20-24, wherein the sample comprises cells or nuclei and the analytes labeled in step (a) are proteins, DNA, cDNA or RNA.

26. The method of any of claims 20-25, wherein step (a) is done by ligation or by hybridization.

27. The method of any of claims 20-25, wherein step (b) is done by a biorthogonal addition reaction.

28. The method of any of claims 20-27, wherein each subunit is an oligonucleotide, a nucleotide, a unique molecular index (UMI), fluorescent dye, or mass tag.

29. A device for collecting a sample from multi-well plate, comprising:
a lip that is adapted to mate with the multiwell plate;
a wall that defines a funnel; and
a cylindrical reservoir at the bottom of the funnel, distal to the lip.

30. The device of claim 29, wherein the cylindrical reservoir has a V- or U-shaped bottom.

31. The device of any of claims 29-30, wherein the device is made from a transparent plastic.

32. The device of any of claims 29-31, wherein the device is made from a single piece of molded plastic.

33. The device of any of claims 29-32, wherein the device comprises foldable supports that, when folded, allows the device to stand autonomously or function as an adapter.

34. The device of any of claims 29-33, wherein the wall that defines a funnel is composed of a radius and slope on both the long and short edges.

35. The device of claim 34, wherein the slope angle, relative to the horizontal, can be any angle from 5° to 35°.

36. The device of any of claims 34-35, wherein the radius of the profile can be any radius from 25 mm to 50 mm.

37. The device of any of claims 34-36, wherein the radius start angle, relative to the vertical, can be any angle from 0° to 25°.

38. The device of any of claims 30-37, wherein the V- or U-shaped bottom is highly polished on both the interior and exterior to allow clear inspection of any sample in that reservoir.

39. A method for collecting a sample from multi-well plate, comprising:
mating a device of any of claims 29-38 with a multi-well plate;
applying a centrifugal force to push the sample from the plate into reservoir via the funnel, thereby collecting the sample in the reservoir.

40. The method of claim 39, wherein the centrifugal force is in the range of 50-500 G.

41. A design of a device for collecting cells or nuclei, that comprises:
a. a conical reservoir for collection; and
b. sloped angle to facilitate pooling;
wherein the device mates with standard SBS footprint plates.
